(19) United States
(12) Patent Application Publication
(10) Pub. No.: US 2024/0219378 A1
(43) Pub. Date: Jul. 4, 2024

(54) METHOD FOR DETECTING EXOPOLYSACCHARIDE

(71) Applicant: MEIJI CO., LTD., Tokyo (JP)

(72) Inventors: Toshifumi OHSHIMA, Tokyo (JP); Kumiko TAKABAYASHI, Tokyo (JP); Kai NAKAYAMA, Tokyo (JP); Akihiro NAKAJIMA, Tokyo (JP)

(73) Assignee: MEIJI CO., LTD., Tokyo (JP)

(21) Appl. No.: 18/286,680

(22) PCT Filed: Mar. 31, 2022
(86) PCT No.: PCT/JP2022/016530

§ 371 Date: Oct. 12, 2023

(30) Foreign Application Priority Data
Apr. 13, 2021 (JP) 2021-067952
Aug. 13, 2021 (JP) 2021-131848

(51) Int. Cl. G01N 33/53; A23C 9/123; C12N 1/20; C12R 1/225

(52) U.S. Cl. CPC G01N 33/5308; A23C 9/123; C12N 1/205; A23V 2400/123; C12R 2001/225; G01N 2333/42; G01N 2400/10

(57) ABSTRACT

The present invention relates to a method capable of efficiently detecting an exopolysaccharide, and, in particular, a method for detecting an exopolysaccharide comprising contacting a sample containing an exopolysaccharide (EPS) with (i) a lectin capable of binding specifically to the exopolysaccharide and (ii) a labeled lectin capable of binding specifically to the exopolysaccharide, and detecting an exopolysaccharide bound to both the lectin of (i) and the labeled lectin of (ii), using a label of the labeled lectin.

### TECHNICAL FIELD

The present invention relates to a method for detecting an exopolysaccharide.

### BACKGROUND ART

Various microorganisms including lactic acid bacteria are known to produce exopolysaccharides (EPSs) and secrete EPSs extracellularly. Typically, an exopolysaccharide is also present in fermented milk, including yogurt, which is produced by fermenting milk with the use of Lactobacillus delbrueckii subsp. bulgaricus (which is also referred to as L. bulgaricus bacterium) and Streptococcus thermophilus (which is also referred to as S. thermophilus bacterium). In recent years, an EPS has drawn attention since it has a variety of physiologically active functions, such as immunostimulatory functions.

An exopolysaccharide in a culture supernatant of microorganisms or fermented milk has been conventionally measured by the phenol-sulfuric acid method (e.g., Non-Patent Document 1). By the phenol-sulfuric acid method, however, all sugars including a monosaccharide, an oligosaccharide (with 2 to 10 sugar residues), and a polysaccharide are detected. Therefore, when quantifying an exopolysaccharide using the phenol-sulfuric acid method, it is necessary to perform a pretreatment in order to completely remove sugars other than the exopolysaccharide, such as a monosaccharide or disaccharide contained in a sample, by ethanol precipitation or other methods. This complicates a procedure for measurement of an exopolysaccharide by the phenol-sulfuric acid method, the measurement takes a long time, and is likely to result in different measurements between manipulative techniques depending on the individual. Accordingly, a method for efficiently measuring an exopolysaccharide has been awaited.

In the phenol-sulfuric acid method, hydrolysis of polysaccharides is caused. To date, a method for measuring a polysaccharide while retaining its structure has not yet been established.

Patent Document 1 discloses an immunoassay method comprising measuring the amount of a compound (e.g., a tumor marker) having a sugar chain in a biological sample by a sandwich immunoassay method using a labeled lectin and an antibody (a ligand) which specifically recognize the sugar chain, wherein a sugar chain compound that competes with a sugar chain of impurities for binding with the labeled lectin is added to reduce a background noise from sugar chains of impurities, such as glycolipids in the biological sample. According to this method, however, it is difficult to detect a polysaccharide such as an exopolysaccharide separately from other sugars such as monosaccharides or oligosaccharides.

Patent Document 2 discloses a method for determining an undifferentiated status of a stem cell with the use of rBC2LCN that is a lectin specific to a undifferentiation sugar chain marker Fucα1-2Galβ1-3GcNAc/GalNAc, which is capable of discriminating an undifferentiated cell from a differentiated cell. Patent Document 2 discloses that the undifferentiation sugar chain marker is detected by a lectin-lectin sandwich method. In the method of Patent Document 2, however, detection of an exopolysaccharide is not expected.

### CITATION LIST

### Patent Document

Patent Document 1: International Publication WO 2014/103553
Patent Document 2: International Publication WO 2013/065302

### Non-Patent Document

Non-Patent Document 1: Ziadi et al., BioMed. Research International, 2018, Vol. 2018, doi: 10.1155/2018/1896240.

### SUMMARY OF THE INVENTION

### Technical Problem to be Solved by the Invention

It is a problem of the present invention to provide a method capable of efficiently detecting an exopolysaccharide.

### Solution to Problem

The present inventors have conducted concentrated studies in order to solve the above problem. As a result, they found that an exopolysaccharide could be detected efficiently by sandwiching the exopolysaccharide with lectins that bind specifically to the exopolysaccharide (i.e., binding the lectins to the exopolysaccharide to sandwich it). This has led to the completion of the present invention.

Specifically, the present invention includes the following.
[1]A method for detecting an exopolysaccharide comprising:
   contacting a sample containing an exopolysaccharide (EPS) with (i) a lectin capable of binding specifically to the exopolysaccharide and (ii) a labeled lectin capable of binding specifically to the exopolysaccharide and
   detecting an exopolysaccharide bound to both the lectin of (i) and the labeled lectin of (ii) using a label of the labeled lectin.
[2] The method according to [1], wherein the sample contains a culture or a fraction thereof comprising a microorganism having an ability to produce the exopolysaccharide.
[3] The method according to [2], wherein the microorganism is a lactic acid bacterium or a bifidobacterium.
[4] The method according to any of [1] to [3], wherein the exopolysaccharide is from a lactic acid bacterium or a bifidobacterium.
[5] The method according to [3] or [4], wherein the lactic acid bacterium is Lactobacillus delbrueckii subsp. bulgaricus OLL1073R-1 strain (Accession Number: FERM BP-10741).
[6] The method according to any of [1] to [5], wherein the labeled lectin of (ii) comprises a lectin that is the same as the lectin of (i).
[7] The method according to any of [1] to [6], wherein the lectin of (i) and the labeled lectin of (ii) bind specifically to a sugar in the exopolysaccharide.
[8] The method according to [7], wherein the sugar is or comprises galactose or glucose.
[9] The method according to any of [1] to [8], wherein the lectin of (i) and the labeled lectin of (ii) are RCA120 or concanavalin A.
[10] The method according to any of [1] to [9], wherein the lectin of (i) is immobilized on a solid carrier.
[11] The method according to [10], wherein the solid carrier is washed before the detection.
[12] The method according to [2], wherein the culture is a fermented food.
[13] The method according to [12], wherein the fermented food is fermented milk.
[14] The method according to any of [1] to [13], which is a method for quantitatively detecting an exopolysaccharide comprising determining the content of an exopolysaccharide in a sample based on the results of the detection of the exopolysaccharide.
[15] A method for producing a fermented food comprising:
   performing fermentation under conditions suitable for increasing an exopolysaccharide, while quantifying an exopolysaccharide by the method according to [14], and increasing the content of the exopolysaccharide in a fermented food.

This description includes the contents as disclosed in the descriptions and drawings of Japanese Patent Application Nos. 2021-067952 and 2021-131848, which are priority applications of the present application.

### Effects of the Invention

According to the present invention, an exopolysaccharide can be efficiently detected.

### BRIEF DESCRIPTION OF DRAWINGS

FIG. 1 is a schematic diagram illustrating a typical embodiment of the method for detecting EPS according to the present invention.

FIG. 2 shows an EPS calibration curve prepared using ConA on a plate prepared with the use of different buffers, and an EPS standard. In FIG. 2, “PBS” (open circle) denotes an EPS calibration curve prepared when using PBS (pH 7.2) as a buffer, “Tris” (shaded triangle) denotes an EPS calibration curve prepared when using 50 mM Tris-HCl buffer (pH 7.2) as a buffer, and “HEPES” (filled square) denotes an EPS calibration curve prepared when using 0.1 M HEPES buffer (pH 8.0) as a buffer.

FIG. 3 shows an EPS calibration curve prepared by assays using an EPS standard and ConA.

FIG. 4 shows an EPS calibration curve prepared by assays using an EPS standard and RCA 120.

FIG. 5 shows an EPS calibration curve prepared by assays using an EPS standard and ConA.

FIG. 6 shows an EPS calibration curve prepared by assays using an EPS standard and ConA.

### EMBODIMENTS OF THE INVENTION

Hereafter, the present invention is described in detail.

The present invention relates to a method for detecting an exopolysaccharide (EPS) using lectins capable of binding specifically to the exopolysaccharide. More specifically, the present invention relates to a method for detecting an exopolysaccharide, characterized by sandwiching an exopolysaccharide with a lectin capable of binding specifically to EPS and a labeled lectin capable of binding specifically to EPS. The method of the present invention basically uses a lectin-lectin sandwich method. The lectin-lectin sandwich method is an assay technique comprising contacting a target substance having a sugar or sugar chain with a lectin having a binding ability to the sugar or sugar chain, thereby sandwiching the target substance with a plurality of the lectins to form a conjugate of the target substance and the lectins bound thereto; and detecting the conjugate. In a typical example of the lectin-lectin sandwich method, a lectin immobilized on a solid carrier and a labeled lectin are used to sandwich a target substance with them, and then a label of the labeled lectin is used to detect a conjugate of the target substance and the lectins bound thereto.

An exopolysaccharide (EPS) is a polysaccharide that is produced by a microorganism and then secreted extracellularly. An exopolysaccharide as a target substance to be detected in the method of the present invention may be derived from any microorganism having an ability to produce an exopolysaccharide. The exopolysaccharide is preferably derived from bacteria, and more preferably derived from a bifidobacterium or a lactic acid bacterium. Examples of the bifidobacterium include, but are not limited to, Bifidobacterium bacteria. Examples of the lactic acid bacterium include, but are not limited to, Lactobacillus bacteria, Laclococcus bacteria, Leuconostoc bacteria, Enterococcus bacteria, Pediococcus bacteria, Tetragenococcus bacteria, and Streptcoccus bacteria. More specific examples of the bifidobacterium include, but are not limited to, Bifidobacterium bifidum, Bifidobacterium longum, and Bifidobacterium lactis. More specific examples of the lactic acid bacterium include, but are not limited to, Lactobacillus delbrueckii subsp. bulgaricus, Lactobacillus casei, Lactobacillus gasseri, Lactobacillus fermentum, Lactobacillus helveticus, Lactobacillus acidophilus, Lactobacillus plantarum, Lactobacillus rhamnosus, Lactobacillus pentosus, Lactobacillus paracasei, Lactobacillus kefiranofaciens, Lactobacillus sake, Lactococcus lactis ssp. lactis, Lactococcus lactis ssp. cremoris, Lactococcus lactis subsp. lactis var. diacetylactis, Leuconostoc mesenteroides, Enterococcus foecahs, Pediococcus pentosaceus, Tetragenococcus halophilus, and Streptococcus thermophilus. The term “Lactobacillus bacteria” refers to lactic acid bacteria that belong to any genus of the 25 genera newly established by reclassification of lactic acid bacteria of conventional genus Lactobacillus, as reported in the literature of Zheng et al., “A taxonomic note on the genus Lactobacillus: Description of 23 novel genera, emended description of the genus Lactobacillus beijerinck 1901, and union of Lactobacillaceae and Leuconostocaceae”, INTERNATIONAL JOURNAL OF SYSTEMATIC AND EVOLUTIONARY MICROBIOLOGY, Volume 70, Issue 4, pp. 2782-2858, published on Apr. 15, 2020; i.e., the genus Lactobacillus, the genus Paralactobacillus, the genus Holzapfelia, the genus Amylolactobacillus, the genus Bombilactobacillus, the genus Companilactobacillus, the genus Lapidilactobacillus, the genus Agrilactobacillus, the genus Schleiferilaclobacillus, the genus Loigolaclobacilus, the genus Lacticaseibacillus, the genus Latilactobacillus, the genus Dellaglioa, the genus Liquorilactobacillus, the genus Ligilactobacillus, the genus Lactiplantibacillus, the genus Furfurilactobacillus, the genus Paucilactobacillus, the genus Limosilactobacillus, the genus Fructilactobacillus, the genus Acetilactobacillus, the genus Apilactobacillus, the genus Levilactobacillus, the genus Secundilactobacillus, and the genus Lentilaclobacillus.

A more preferred example of the lactic acid bacterium is Lactobacillus delbrueckii subsp. bulgaricus, and particularly preferably Lactobacillus delbrueckii subsp. bulgaricus OLL1073R-1 strain. Other examples of the lactic acid bacterium include bacterial strains described in Examples below.

Lactobacillus delbrueckii subsp. bulgaricus OLL1073R-1 strain is deposited internationally under the Budapest Treaty with the International Patent Organism Depositary, the National Institute of Technology and Evaluation (NITE-IPOD) (the former name: the International Patent Organism Depositary, the National Institute of Advanced Industrial Science and Technology) (#120, 2-5-8 Kazusakamatari, Kisarazu-shi, Chiba, 292-0818, Japan) as of Feb. 22, 1999 (the date of the original deposit), under Accession Number FERM BP-10741. This deposited strain was transferred from the domestic deposit (the original deposit) to the international deposit under the Budapest Treaty on Nov. 29, 2006. The current depositor of Lactobacillus delbrueckii subsp. bulgaricus OLL1073R-1 strain is Meiji Co., Ltd.

In general, exopolysaccharides are broadly classified into homo and hetero types based on a difference in their constituent sugars. An exopolysaccharide to be detected by the method of the present invention may be a homo-exopolysaccharide or a hetero-exopolysaccharide. The exopolysaccharide may have a branched structure (a side chain). As major constituent sugars of a homo-exopolysaccharide, in general, glucose and fructose are known. As major constituent sugars of a hetero-exopolysaccharide, in particular, a hetero-exopolysaccharide derived from a lactic acid bacterium or a bifidobacterium, in general, galactose, glucose, mannose, and rhamnose are known. In general, a hetero-exopolysaccharide, in particular, a hetero-exopolysaccharide derived from a lactic acid bacterium or a bifidobacterium comprises at least one of galactose and glucose as a constituent sugar, and many of such hetero-exopolysaccharides comprise galactose and glucose as constituent sugars. In one embodiment, an exopolysaccharide to be detected by the method of the present invention may comprise galactose; galactose and glucose; or glucose. In one embodiment, an exopolysaccharide to be detected by the method of the present invention may comprise galactose; galactose and glucose; or glucose, in the main chain and/or side chain. Galactose as a constituent sugar of an exopolysaccharide may be α-galactose (α-Gal) or β-galactose (β-Gal). Glucose as a constituent sugar of an exopolysaccharide may be α-glucose (α-Glc) or β-glucose (β-Glc). In one embodiment, an exopolysaccharide to be detected by the method of the present invention may comprise mannose and/or rhamnose in the main chain and/or side chain. For example, the exopolysaccharide may comprise mannose and/or rhamnose, in addition to galactose and/or glucose, in the main chain and/or side chain. In another embodiment, an exopolysaccharide to be detected by the method of the present invention may comprise at least one selected from the group consisting of galactose, glucose, and mannose, in the main chain and/or side chain. In one embodiment, an exopolysaccharide to be detected by the method of the present invention may comprise a sugar other than galactose, glucose, mannose, and rhamnose, in the main chain and/or side chain, and the exopolysaccharide may comprise such sugar, in addition to galactose, glucose, mannose, and/or rhamnose, in the main chain and/or side chain. It should be noted that an exopolysaccharide to be detected by the method of the present invention is not limited to those exemplified above.

In the present invention, for detecting an exopolysaccharide, (i) a lectin capable of binding specifically to the exopolysaccharide (hereafter, it may also be referred to as “the first lectin”) and (ii) a lectin which is labeled (a labeled lectin) and capable of binding specifically to the exopolysaccharide (hereafter, it may also be referred to as “the second lectin”) can be used.

The present invention provides a method for detecting an exopolysaccharide comprising contacting a sample containing an exopolysaccharide (EPS) with (i) a lectin capable of binding specifically to the exopolysaccharide and (ii) a labeled lectin capable of binding specifically to the exopolysaccharide, and detecting an exopolysaccharide bound to both the lectin of (i) and the labeled lectin of (ii) using a label of the labeled lectin. In one embodiment, an exopolysaccharide bound to both the lectin of (i) and the labeled lectin of (ii) may be separated from the sample, before detection.

A lectin is a protein (excluding an antibody and an antibody fragment) that has a specific binding ability to a sugar or sugar chain. A wide variety of lectins have been found and sugar specificity of the lectins (lectin-binding sugars) has also been found. A lectin capable of binding specifically to an exopolysaccharide, which is used in the method of the present invention, may be a lectin capable of binding specifically to a sugar (a sugar residue) in the main chain and/or side chain, for example, a sugar (a sugar residue) in the side chain, of an exopolysaccharide to be detected by the method of the present invention. A sugar (a sugar residue) to which the lectin has a binding ability is not particularly limited; but in one embodiment, the sugar may be one or two or more selected from the group consisting of galactose (α-Gal or β-Gal), glucose (α-Glc or β-Glc), mannose (e.g., α-Man), N-acetylglucosamine (GlcNAc), N-acetylgalactosamine (GalNAc), and glucuronic acid (GlcA), or a sugar moiety comprising at least one selected from the group consisting of galactose, glucose, mannose, N-acetylglucosamine, N-acetylgalactosamine, and glucuronic acid (e.g., an oligosaccharide sequence such as a disaccharide). In one embodiment, the lectin may be specific to at least one selected from the group consisting of galactose, glucose, and mannose. For example, such lectin may be a lectin that is specific to galactose, or a lectin that is specific to mannose and glucose. In one embodiment, the lectin may be specific to at least one sugar selected from the group consisting of galactose, glucose, and mannose in an exopolysaccharide. In one embodiment, the lectin may be an R-type lectin, an L-type lectin, or an M-type lectin. The lectin may be derived from a plant (e.g., Ricinus communis, or a leguminous plant) or an animal. Specific examples of the lectins include, but are not particularly limited to, RCA120 exhibiting specific binding to galactose and concanavalin A (which may also be referred to as “ConA”) exhibiting specific binding to α-glucose and α-mannose. RCA120 is known as an example of the R-type lectin, and concanavalin A is known as an example of the L-type lectin. Each of RCA120 and concanavalin A can be suitably used to detect an exopolysaccharide derived from, in particular, a lactic acid bacterium or a bifidobacterium, preferably a lactic acid bacterium, and more preferably Lactobacillus bacteria.

In the present invention, lectins having the same or different sugar specificity may be used as (i) a lectin capable of binding specifically to the exopolysaccharide (the first lectin) and (ii) a labeled lectin capable of binding specifically to the exopolysaccharide (the second lectin). However, use of lectins having the same sugar specificity as the lectins of (i) and (ii) is preferred. In the present invention, (ii) a labeled lectin capable of binding specifically to the exopolysaccharide (the second lectin) preferably comprise a lectin that is the same as (i) a lectin capable of binding specifically to the exopolysaccharide (the first lectin).

The first lectin and the second lectin may each be a single lectin or a mixture of a plurality of types of lectins. The first lectin and the second lectin may each be a recombinant, which is produced using genetically engineering techniques in prokaryotic or eukaryotic cells, a naturally occurring lectin isolated from a biological material, or a genetically modified mutant. The first lectin and the second lectin may or may not have a sugar chain. In one embodiment, when the first lectin comprises a lectin that is of a different type from that of the second lectin, it is preferred that either or both the first lectin and the second lectin do not have a sugar chain.

The labeled lectin, which is the second lectin, is labeled by attaching a label (a label substance) to or incorporating it into a lectin. The label is not particularly limited, provided that it can be used for protein detection. Examples of the label include an enzyme (e.g., peroxidase), a coenzyme, biotin, avidin, streptavidin, a radioisotope, a fluorescent substance (e.g., a fluorescent dye or a fluorescent protein), a chemiluminescent substance, and an ultraviolet absorber, such as labels that are used in immunoassays. In one embodiment, the lectin may be linked to the label directly or via a linker. The labeled lectin may be a fusion protein of a labeling protein such as a fluorescent protein and a lectin. A lectin can be labeled by a conventional method.

In the method of the present invention, a sample containing an exopolysaccharide (EPS) is contacted with the first lectin and the second lectin (the labeled lectin). In general, a sample containing an exopolysaccharide is a liquid sample. In one embodiment, a sample containing an exopolysaccharide may be a supernatant fraction obtained by a separation method such as centrifugation (e.g., at 12,000 g for 5 to 15 minutes) or separation by filtration, or a dilution thereof. In one embodiment, a sample containing an exopolysaccharide may be a precipitation fraction generated by alcohol precipitation (e.g., ethanol precipitation) or a dilution thereof (e.g., a diluted solution in a buffer such as a HEPES buffer). In one embodiment, a sample containing an exopolysaccharide may be a precipitation fraction generated by subjecting a supernatant fraction obtained by centrifugation (e.g., at 12,000 g for 5 to 15 minutes) or separation by filtration or a dilution thereof to alcohol precipitation (e.g., ethanol precipitation); or a dilution thereof. In one embodiment, a sample containing an exopolysaccharide may comprise a culture or a fraction thereof comprising a microorganism having an ability to produce the exopolysaccharide. The term “microorganism” used herein is as described above about the microorganism from which EPS is derived. For example, such microorganism may be a lactic acid bacterium or a bifidobacterium, including Lactobacillus bacteria such as Lactobacillus delbrueckii subsp. bulgaricus OLL1073R-1 strain, and Streptococcus bacteria such as Streptococcus thermophilus. The “culture” of a microorganism may be a co-culture of a plurality of strains or a plurality of species of microorganisms. The co-culture may be, for example, a co-culture of Lactobacillus bacteria such as Lactobacillus delbrueckii subsp. bulgaricus, and Streptococcus bacteria such as Streptococcus thermophilus, and/or bifidobacteria.

The term “culture” used herein encompasses a fermented product. Examples of fermented products include, but are not limited to, fermented foods, such as fermented milk (e.g., yogurt, Caspian Sea yogurt, kefir, and Viili), cheese, plant-based fermented products (e.g., plant material-fermented yogurt) prepared with plant materials (e.g., soymilk, almond milk, and coconut milk), pickles, kimchee, soy sauce, and miso (fermented soybean paste); and are preferably fermented milk, cheese, and a plant-based fermented products. The yogurt may be, for example, plain yogurt, hard yogurt, drink-type yogurt, soft yogurt, or frozen yogurt. The fermented product may comprise, in addition to the above-mentioned microorganism and a fermentation substrate, a food raw material or food additive used in the food industry and various substances such as nutrients or additives for culturing. The fermented milk may comprise, as materials other than raw milk, food raw materials or food additives, such as a protein, a cream, a fruit, a monosaccharide, a disaccharide, an oligosaccharide, a sugar alcohol, seeds, a sweetener, a flavor, fruit juice, a gelling agent, or a thickener. The term lactic acid bacteria drink is defined as “a drink (excluding fermented milk) produced by fermenting milk or the like with lactic acid bacteria or yeast and then processing it or using it as one of major raw materials” under the Ministerial Ordinance on Milk and Milk products Concerning Compositional Standards, etc. (abbreviated as “Ministerial Ordinance for Milk, etc.”) based on the Food Sanitation Law of Japan. In the present invention, however, the “fermented milk” encompasses a lactic acid bacteria drink. In the method of the present invention, a known technique may be added as a step of pretreatment of a sample containing EPS, as appropriate, to further improve the detection sensitivity. Specific examples of the pretreatment techniques include, but are not limited to, centrifugation, removal of macromolecules by ultrafiltration, protein precipitation with an organic solvent including alcohol such as ethanol or an acid such as trichloroacetic acid or ammonium sulfate, and protein degradation by enzyme treatment (with e.g., protease, lipase, or pectinase). The fraction of a microbial culture may be any fraction that can comprise an exopolysaccharide (EPS). The fraction may be a non-purified fraction, such as a culture supernatant fraction (e.g., a supernatant fraction obtained by a separation technique such as centrifugation (e.g., at 12,000 g for 5 to 15 minutes) or separation by filtration), a precipitation fraction (e.g., a precipitation fraction generated by alcohol precipitation such as ethanol precipitation), a partially purified fraction, or a sugar fraction. The fraction may or may not be a purified EPS fraction. A sample containing an exopolysaccharide may be a dilution (e.g., a diluted solution in a buffer such as HEPES buffer) or a suspension of a microbial culture or a fraction thereof. In another embodiment, a sample containing an exopolysaccharide may comprise an isolated, purified, or synthetic exopolysaccharide.

In many cases, the sample containing an exopolysaccharide contains a low-molecular weight sugar. For example, such sample contains a low-molecular weight sugar such as a monosaccharide or disaccharide released from an exopolysaccharide, or a low-molecular weight sugar derived from a microorganism or medium used for production of an exopolysaccharide. When the sample containing an exopolysaccharide is allowed to react with a lectin, accordingly, a lectin would react not only with a polysaccharide but also with the low-molecular weight sugar. However, the method of the present invention involving the use of a lectin was found to be capable of detecting an exopolysaccharide with high sensitivity and to be less likely to be influenced by a low-molecular weight sugar contained as an impurity in the sample. The high sensitive detection of an exopolysaccharide with the use of a lectin is considered to be because the exopolysaccharide is a macromolecular compound and thus has a large number of lectin-binding sites per molecule. The result that the detection of the exopolysaccharide using a lectin is less likely to be influenced by a low-molecular weight sugar was surprising and advantageous.

In the method of the present invention, a low-molecular weight sugar is not detected, and thus it is not necessary to perform a pretreatment to remove low-molecular weight sugars from a sample containing an exopolysaccharide. However, in one embodiment of the method of the present invention, for example, in a case where a large quantity of low-molecular weight sugars are present in a sample containing an exopolysaccharide, a pretreatment to separate a polysaccharide from low-molecular weight sugars may be optionally performed by alcohol precipitation (e.g., ethanol precipitation) or the like, followed by reacting the resulting separated fraction (e.g., an alcohol-precipitated fraction) with a lectin according to the method of the present invention, which results in the preparation of better quantitative data.

In a preferred embodiment of the method of the present invention, the first lectin may be immobilized on a solid carrier (support). Alternatively, the first lectin may be configured to enable an immobilization on a solid carrier; for example, the first lectin may be attached to a substance that facilitates immobilization on a solid carrier (support). The solid carrier may be of any form. For example, the solid carrier may be a plate such as a microtiter plate (e.g., a 96-well microtiter plate), a substrate such as a chip or array, a sheet, a thread, a tube, or a particle. The particle may be a fine particle, such as a nanoparticle or microparticle, or a bead particle bigger than the fine particle. The particle may be a magnetic particle. The solid carrier may have at least a lectin-binding region that is composed of an insoluble material suitable for protein immobilization, for example, resin, such as silicon resin, polystyrene resin, or polyacrylamide resin; glass, or metal.

The first lectin, as a so-called ligand, can be immobilized on (bound to) a solid carrier by a conventional method such as chemical binding or physical adsorption. In one embodiment, the first lectin is preferably immobilized on a solid carrier with the use of a buffer. A buffer used for immobilization may be, but is not particularly limited, preferably a HEPES buffer, and more preferably a 0.01 M to 1 M HEPES buffer, and further preferably a 0.05 M to 0.5 M HEPES buffer (pH 7.5 to 8.5). For example, the buffer may be approximately 0.1 M HEPES buffer (pH approximately 8.0). Specifically, a HEPES buffer comprising the first lectin may be contacted with a solid carrier and left for a given period of time, followed by subsequent washing and blocking with a HEPES buffer. The first lectin may be immobilized on a solid carrier with the use of the above-mentioned buffer.

In the method of the present invention, a sample containing an exopolysaccharide (EPS) is contacted with the first lectin and the second lectin (the labeled lectin). Through such contact, the exopolysaccharide in the sample can bind to the first lectin and the second lectin (the labeled lectin). In a typical embodiment, the method of the present invention may comprise contacting a sample containing an exopolysaccharide with the first lectin to bind the exopolysaccharide in the sample to the first lectin, and then contacting the sample with the second lectin to further bind the exopolysaccharide to the second lectin. In one embodiment, a sample containing an exopolysaccharide is added to the first lectin to react therewith, so that the exopolysaccharide in a sample is bound to the first lectin, and subsequently, washing is performed as appropriate to remove free ingredients, prior to contacting the exopolysaccharide with the second lectin. After washing, the second lectin is added for reaction to the exopolysaccharide bound to the first lectin, so that the exopolysaccharide bound to the first lectin can further be bound to the second lectin. Washing can be performed with the use of any wash solution. In general, a buffer (e.g., a HEPES buffer or PBS) containing a surfactant (e.g., Tween 20) can be used as a wash solution.

In one embodiment of the method of the present invention, it is not necessary to perform a pretreatment or posttreatment to purify an exopolysaccharide in a sample, such as protein removal, before or after contacting the sample containing an exopolysaccharide with a lectin. Accordingly, in one embodiment, the method of the present invention does not necessarily comprise a step of purifying an exopolysaccharide.

Subsequently, the exopolysaccharide that have bound to both the first lectin and the second lectin (the labeled lectin) may be detected using a label of the labeled lectin. Detection using a label of the labeled lectin (e.g., detection of a signal from a fluorescent, luminescent, chromogenic, or radioactive label) can be performed in accordance with a conventional technique. When a fluorescent substance is used as a label, for example, a fluorescence can be detected by visual or microscopic observation or by measuring the absorbance at the wavelength suitable for fluorescence detection with the use of a spectrophotometer or the like. When a chemiluminescent substance is used as a label, a chemiluminescence can be detected by visual or microscopic observation or by measuring the absorbance at the wavelength suitable for chemiluminescence detection with the use of a spectrophotometer or the like. When an enzyme label, such as peroxidase, is used, the label is reacted with a chromogenic substrate, such as TMB (3,3′,5,5′-tetramethylbenzidine) for peroxidase, the developed color is observed or measured with the use of a spectrophotometer or the like, and detection can be thus performed.

The exopolysaccharide that have bound to both the first lectin and the second lectin (the labeled lectin) is preferably bound to a solid carrier via the first lectin before detection. If the exopolysaccharide that have bound to both the first lectin and the second lectin (the labeled lectin) is not bound to a solid carrier, the exopolysaccharide can be bound to the solid carrier, for example, using a substance that facilitates immobilization on the solid carrier bound to the first lectin. In addition, it is preferred that the solid carrier be washed before detection to eliminate free components. If the exopolysaccharide that have bound to both the first lectin and the second lectin (the labeled lectin) is bound to the solid carrier, the solid carrier may be washed, so that many of impurities in the sample can be easily eliminated from the test system. While a solid carrier can be washed using any wash solution, a buffer (e.g., a HEPES buffer or PBS) containing a surfactant (e.g., Tween 20) can be generally used as a wash solution. By the method comprising washing the solid carrier to which the exopolysaccharide has bound via the first lectin, the exopolysaccharide can be efficiently separated from the sample, before detection.

In the method of the present invention, detection using a label of the labeled lectin is performed as described above, so that the exopolysaccharide that have bound to both the first lectin and the second lectin (the labeled lectin) in the sample can be detected. The method of the present invention may be a method of quantitative detection (a quantification method) or a method of qualitative detection. The present invention also relates to a method of quantitative detection of an exopolysaccharide (or a method for quantifying an exopolysaccharide) comprising determining the content of an exopolysaccharide in a sample based on the results of detection of the exopolysaccharide.

The content of an exopolysaccharide in a sample can be determined based on the results of the detection of the exopolysaccharide in the sample using conventional techniques. Typically, using an EPS calibration curve (e.g., the EPS calibration curve prepared in Examples below) which is prepared through the detection of an exopolysaccharide standard (a purified exopolysaccharide of a known concentration; which is preferably of an exopolysaccharide derived from the same microorganism as the exopolysaccharide in the sample) in the same way as the detection of the exopolysaccharide in the sample with the use of the first lectin and the second lectin (the labeled lectin), the content of the exopolysaccharide in the sample (e.g., the concentration of the exopolysaccharide in the sample) can be determined based on the results of the detection of the exopolysaccharide for the sample.

In the method of the present invention, the content of an exopolysaccharide in a sample can be measured with high sensitivity and high stability without a pretreatment or posttreatment, such as protein removal.

The method of the present invention may comprise, prior to the step of contacting a sample containing an exopolysaccharide with the first lectin and the second lectin (the labeled lectin), a step of identifying or screening for a lectin capable of binding specifically to the exopolysaccharide. The step of identifying (or screening) may comprise, for example, adding a purified exopolysaccharide to a lectin array and selecting a lectin bound specifically to the exopolysaccharide.

Hereafter, the method of the present invention is described with reference to typical steps.

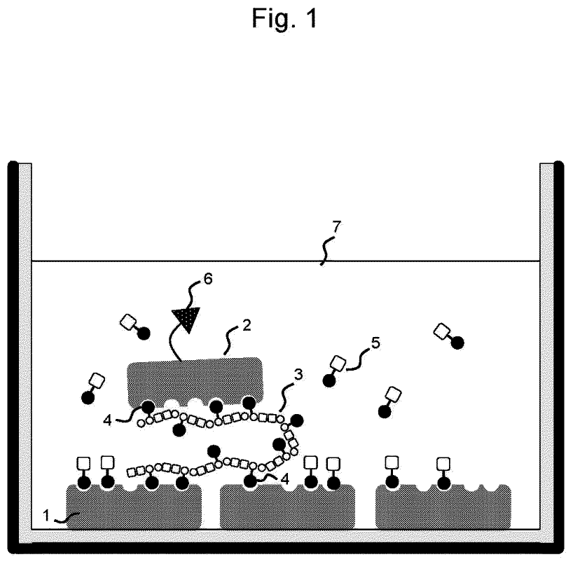

In one embodiment, the method of the present invention can be performed with the use of, for example, the reaction system as illustrated in FIG. 1. The method of the present invention can be performed, for example, by the following procedures 1) to 6), when using a label that enables the enzymic reaction-based detection using a detection substrate.
1) A buffer comprising the first lectin (“1” in FIG. 1) (e.g., a lectin solution with a concentration of 1 to 100 μg/ml, or 10 to 20 μg/ml) is added to a microtiter plate, and incubated (e.g., at 4° C. overnight) to immobilize the first lectin on the microtiter plate. The buffer comprising the first lectin is preferably the HEPES buffer as described above, but the buffer is not limited thereto.
2) Diluted exopolysaccharide standards in a buffer, and a sample containing an exopolysaccharide (“7” in FIG. 1) are each separately added to a microtiter plate to allow an exopolysaccharide (“3” in FIG. 1) to react with the first lectin (e.g., by incubation at 30° C. to 40° C. for 1 to 3 hours). The exopolysaccharide is bound to the first lectin via a lectin-binding sugar (“4” in FIG. 1).
3) The microtiter plate is washed, and then the second lectin (the labeled lectin) (“2” in FIG. 1) is added. As the labeled lectin, for example, a biotin-labeled lectin or a peroxidase-labeled lectin is used. In such a case, biotin and peroxidase are each a label of the lectin (“6” in FIG. 1). The labeled lectin binds to the exopolysaccharide via a lectin-binding sugar (“4” in FIG. 1).
4) The microtiter plate is washed, and a detection substrate is added. As an example, if a biotin-labeled lectin is added in procedure 3), peroxidase-labeled streptavidin or peroxidase-labeled avidin, and TMB (3,3′,5,5′-tetramethylbenzidine), which is a chromogenic substrate of peroxidase, can be used as detection substrates. As another example, if a peroxidase-labeled lectin is added in procedure 3), TMB (3,3′,5,5′-tetramethylbenzidine), which is a chromogenic substrate of peroxidase, can be used as a detection substrate. When such chromogenic substrate is used, it develops color in the presence of an exopolysaccharide. An impurity sugar (“5” in FIG. 1, such as a disaccharide or monosaccharide such as lactose, or a low-molecular-weight sugar having a molecular weight of up to about several thousands), which is not sandwiched between the first lectin and the second lectin, would not be detected.
5) After the stopping of a detection reaction such as a chromogenic reaction, the absorbance is measured at an adequate wavelength at which the reaction product can be detected (e.g., at 450 nm if TMB is used as a detection substrate).
6) A calibration curve is prepared based on the measurements of absorbance obtained using the exopolysaccharide standard, and then the content of the exopolysaccharide (the concentration of the exopolysaccharide) in the sample is determined on the basis of the measurements of absorbance of the sample, using the calibration curve.

In the present invention, detection can be performed with high specificity with the use of a lectin capable of binding specifically to a constituent sugar in an exopolysaccharide, which depends on a type of the exopolysaccharide to be measured. In the present invention, a reaction in which a sugar is sandwiched between lectins can be used to selectively detect and quantify an exopolysaccharide which is a macromolecular substance having a large number of lectin-binding sites. Therefore, in many cases, even if compounds including a low-molecular weight sugar such as a monosaccharide or disaccharide are present in a test system, the reactivity of such compounds with a lectin would be only very low and would not substantially affect measurement of the exopolysaccharide. Accordingly, the method of the present invention enables measurement of an exopolysaccharide in a simple method within a short period of time without a treatment to remove sugar other than the exopolysaccharide. In addition, a plate with many wells, such as a 96-well microtiter plate, can be used for measurement of many specimens at a time.

The method of the present invention utilizes the reaction specificity between a lectin and a polysaccharide. In a preferred embodiment, accordingly, an exopolysaccharide can be quantified at as small as several hundred ng/ml of exopolysaccharide, which enables a measurement of exopolysaccharide with sensitivity that is at least about 100 times higher than the sensitivity of the phenol-sulfuric acid method.

In addition, the present invention relates to a method for producing a fermented food involving the use of the method for detecting an exopolysaccharide, during production of the fermented food. The present invention provides a method for producing a fermented food comprising fermenting a food raw material, and preferably performing fermentation under conditions suitable for increasing an exopolysaccharide, while quantifying an exopolysaccharide by the above-mentioned method for detecting an exopolysaccharide. In the present invention, for example, a food raw material (fermentation substrate) can be fermented while quantifying an exopolysaccharide by the above-mentioned method for detecting an exopolysaccharide. In such a case, it is preferred that fermentation be performed under conditions suitable for increasing an exopolysaccharide, thereby increasing the content of an exopolysaccharide in a fermented food. While a person skilled in the art can adequately determine the conditions suitable for increasing an exopolysaccharide, using the above-mentioned method for detecting an exopolysaccharide, or the like; for example, fermentation conditions under which an increase over time in the amount of an exopolysaccharide can be observed may be employed. For example, the method of the present invention may be a method for producing a fermented food comprising fermenting a food raw material while quantifying an exopolysaccharide by the above-mentioned method for detecting an exopolysaccharide and checking an increase over time in the amount of an exopolysaccharide, thereby increasing the content of an exopolysaccharide in the fermented food. In the method for producing a fermented food according to the present invention as described above, it is possible to monitor or confirm that, with the use of the result of the detection according to the above-mentioned method for detecting an exopolysaccharide, as an indicator, the content of an exopolysaccharide in a fermented food would be increased to a given or preferred level, or would be increased at a given or preferred rate. The present invention also relates to a fermented food obtained by the method for producing a fermented food.

### EXAMPLES

Hereafter, the present invention is described in greater detail with reference to Examples, although the technical scope of the present invention is not limited to these Examples.

### Example 1

### 1) Preparation of Exopolysaccharide (EPS)

EPS in a culture obtained by culturing Lactobacillus delbrueckii subsp. bulgaricus OLL1073R-1 strain (Accession Number: FERM BP-10741) in a 10 mass % skim milk medium was purified. Specifically, the OLL1073R-1 strain was cultured in the above-mentioned medium at 37° C. for 18 hours, trichloroacetic acid was added to the resulting culture to the final concentration of 10 mass %, and the denatured protein was removed. Then, cold ethanol was added thereto, allowed to stand at 4° C. until the following day, and a precipitate containing EPS was obtained. The precipitate was dialyzed against Milli-Q® water (ultrapure water) using a dialysis membrane (molecular weight cut off 6,000 to 8,000), and subjected to enzymatic degradation of nucleic acids and proteins, and ethanol precipitation was then performed again to obtain a precipitate. In order to purify EPS, the precipitate was dissolved in Milli-Q® water, dialyzed again, and lyophilized. Thus, an EPS standard was prepared.

### 2) Examination of Lectin

Various lectins (Table 1) prepared at 10 μg/ml in phosphate buffered saline (PBS) (pH 7.2) were added at 100 μl/well to a microtiter plate, and the microtiter plate was left at 4° C. overnight for immobilization of the lectins. Subsequently, the microtiter plate was washed two times with PBS, PBS supplemented with 1% BSA was added at 200 μl/well, and the microtiter plate was left at 4° C. overnight, for blocking the plate. The microtiter plate was washed two times with a wash solution (PBS containing 0.05% Tween 20; the same applies below in this Example), a sample of the above-mentioned EPS standard prepared at a concentration of 0.1 to 10 μg/ml in PBS supplemented with 0.1% BSA was added thereto at 100 μl/well, and incubated for reaction at 37° C. for 2 hours. Subsequently, the microtiter plate was washed three times with the wash solution, a biotin-labeled lectin (in which a lectin of the same type as used for immobilization is used) prepared at 0.5 μg/ml in a diluent (PBS supplemented with 0.1% BSA) was added thereto at 100 μl/well, and incubated at 37° C. for 1 hour. After the microtiter plate was washed three times with the wash solution, peroxidase-labeled streptavidin was added, and incubated for reaction at 37° C. for 1 hour. After the microtiter plate was washed three times with the wash solution, chromogenic substrate TMB (3,3′,5,5′-tetramethylbenzidine; KPL SureBlue™), was added thereto at 100 μl/well, and incubated at room temperature for 10 minutes to produce a reaction to develop color. Then, 1 M hydrochloric acid was added at 100 μl/well to stop the reaction, and the absorbance at 450 nm was measured using a microplate reader. When the measured value was equivalent to that of the blank (diluent. PBS supplemented with 0.1% BSA), the lectin was evaluated as “not reacted.” When the measured value was greater than that of the blank, the lectin was evaluated as “reacted.”

Table 1 shows the reactivities between the EPS and lectins shown as described above.

TABLE 1

| | | Reactivity with |
|---|---|---|
| Immobilized and | | OLL1073R-1- |
| labeled lectin | Sugar specificity | derived EPS |
| GSL I-B4* | α-Gal | Not reacted |
| (Griffonia (Bandeiraea) simplicifolia | | |
| Lectin I -Isolectin B4) | | |
| CGL2** | β-Gal, | Not reacted |
| (Coprinopsis cinerea-derived) | GalNAcα1-3Gal [Blood Group A], | |
| | Galα1-3Gal [Blood Group B], | |
| | Poly-LacNAc | |
| RCA120* | Gal | Reacted |
| (Ricinus communis Agglutinin I | | |
| (Castor Bean), seed-derived) | | |
| ConA (concanavalin A)* | α-Man, α-Glc | Reacted |
| (Canavalia ensiformis (Jack Bean) | | |
| seed-derived) | | |

*manufactured by Vector Laboratories
**manufactured by FUJIFILM Wako Pure Chemical Corporation

While the OLL1073R-1-derived EPS reacted with RCA120, the EPS did not react with (not bind to) GSL I-B4 or CGL2. Since the OLL1073R-1-derived EPS has β-Gal (β-galactose) in its side chain, the conformation of β-Gal of the side chain of the EPS was considered to influence the reactivity with the lectin.

Further, the reactivity of guar gum, instead of EPS, with GSL I-B4 was examined in the same method as described above. As a result, guar gum showed strong reactivity with GSL I-B4. GSL I-B4 is considered to have reacted with galactose in the side chain of guar gum. The results indicate that a sugar residue in the side chain of a polysaccharide react readily with a lectin.

The OLL1073R-1-derived EPS was found to comprise glucose as a constituent sugar and to strongly react with (bind to) ConA (concanavalin A) having glucose affinity.

### 3) Examination of Buffer

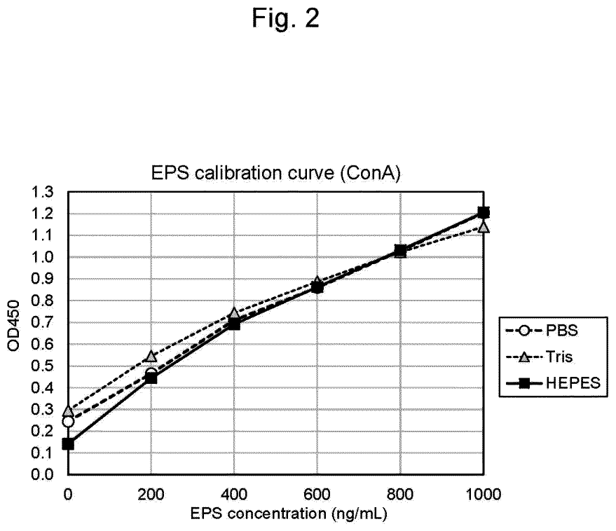

Usability of various buffers was compared and examined. Three buffers: the phosphate buffered saline (PBS) (pH 7.2), and alternatives thereof; i.e., 50 mM Tris-HCl buffer (pH 7.2), and 0.1 M HEPES (4-(2-hydroxyethyl)-1-piperadineethanesulfonic acid) buffer (pH 8.0), were tested. As a lectin, ConA or RCA120 was used. ConA or RCA120 prepared at 10 μg/ml in any of the buffers was used to coat a microtiter plate, and the microtiter plate was then blocked with the buffer used above supplemented with 1% BSA (bovine serum albumin). With the use of the microtiter plates thus prepared, the reactivities between the EPS and the lectins were examined in the same method as in 2) above. In the examination, PBS was used as a diluent or a wash solution. Based on the absorbances measured at 450 nm in the same method as described above, calibration curves were prepared. Concerning ConA, as shown in FIG. 2, the absorbance of the blank was found to be lower on the plate prepared with the use of 0.1 M HEPES buffer (pH 8.0) as a result of comparison among the three types of buffers.

The lectin-immobilized plates prepared with the use of a 0.1 M HEPES buffer (pH 8.0) did not show change in the accuracy of the reaction with the EPS even a month later, which indicates particularly high storage stability of such plates.

### Example 2: Detection of EPS in Culture Supernatant Derived from the OLL1073R-1 Strain

### 1) ConA

### a) Preparation of Microtiter Plate

ConA prepared at 10 μg/ml in 0.1 M HEPES (pH 8.0) was added at 100 μl/well to a microtiter plate, and the plate was left at 4° C. overnight for immobilization of ConA. Subsequently, the microtiter plate was washed two times with 0.1 M HEPES buffer (pH 8.0), and a 0.1 M HEPES buffer (pH 8.0) supplemented with 1% BSA was added at 200 μl/well, and the plate was left at 4° C. overnight, for blocking the plate.

### b) Preparation of Standards and Samples

The EPS standard derived from the OLL1073R-1 strain prepared in Example 1-1) was prepared at 200 to 1,000 ng/ml in a diluent (0.1 M HEPES buffer (pH 8.0) supplemented with 0.1% BSA) to obtain diluted EPS standards.

Lactobacillus delbrueckii subsp. bulgaricus OLL1073R-1 strain was cultured in a 10 mass % skim milk medium at 37° C. for 24 hours, and cells were removed by centrifugation to obtain a culture supernatant. The culture supernatant obtained was used as a sample “without pretreatment.” After trichloroacetic acid (TCA) was added to the obtained culture supernatant to the final concentration of 5%, the mixture was centrifuged at 12,000 g for 20 minutes, and the resulting supernatant was added to a diluent (0.1 M HEPES buffer (pH 8.0) supplemented with 0.1% BSA) to dilute the supernatant to 1,000-fold. The diluted supernatant was used as a sample “with pretreatment.”

c) Detection with ConA

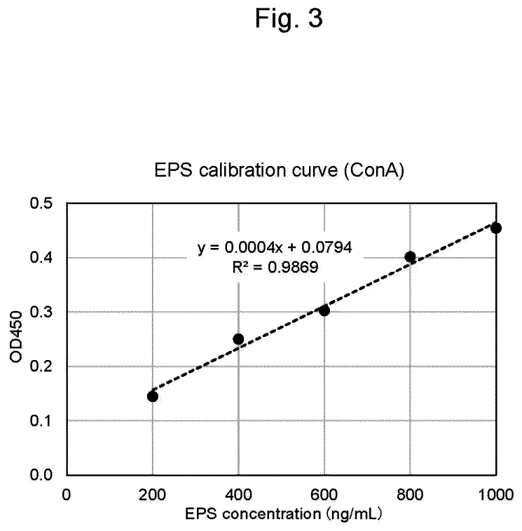

The microtiter plate immobilized with ConA and blocked as described in Example 2-1) a) above was washed two times with a wash solution (0.1 M HEPES buffer (pH 8.0) supplemented with 0.05% Tween 20; the same applies below concerning the detection with ConA in this Example), and then each of the diluted EPS standards or the sample with or without pretreatment as described above was added at 100 dl/well, and incubated for reaction at 37° C. for 30 minutes. After the microtiter plate was washed three times with the wash solution, peroxidase-labeled ConA prepared at 0.5 μg/ml in a diluent (0.1 M HEPES buffer (pH 8.0)) was added thereto at 100 μl/well, and incubated for reaction at 37° C. for 30 minutes. After the microtiter plate was washed three times with the wash solution, chromogenic substrate TMB (KPL SureBlue™) was added thereto at 100 μl/well, and incubated at room temperature for 10 minutes to produce a reaction to develop color. Then, 1 M hydrochloric acid was added at 100 μl/well to stop the reaction, and the absorbance at 450 nm was measured using a microplate reader. A calibration curve was prepared based on the measured values (mean values) obtained with the use of the diluted EPS standards (FIG. 3).

On the basis of the calibration curve shown in FIG. 3, the amount of EPS in the sample was determined based on the measured value obtained with the use of the sample with pretreatment or the sample without pretreatment (Table 2 below).

### 2) RCA120

### a) Preparation of Microtiter Plate

RCA120 prepared at 20 μg/ml in phosphate buffered saline (PBS) (pH 7.8) was added at 100 μl/well to a microtiter plate, and the plate was left at 4° C. overnight for immobilization of RCA120. Subsequently, the microtiter plate was washed two times with phosphate buffered saline (PBS) (pH 7.8), and then phosphate buffered saline (PBS) (pH 7.8) supplemented with 1% BSA was added at 200 μl/well, and the plate was left at 4° C. overnight, for blocking the plate.

### b) Preparation of Standards and Samples

The EPS standard derived from the OLL1073R-1 strain prepared in Example 1-1) was prepared at 200 to 1,000 ng/ml in a diluent (10 mM phosphate buffered saline (pH 7.8) supplemented with 0.1% BSA) to obtain diluted EPS standards.

Lactobacillus delbrueckii subsp. bulgaricus OLL1073R-1 strain was cultured in a 10 mass % skim milk medium at 37° C. for 24 hours, and cells were removed by centrifugation to obtain a culture supernatant. The culture supernatant obtained was used as a sample “without pretreatment.” After trichloroacetic acid (TCA) was added to the obtained culture supernatant to the final concentration of 5%, the mixture was centrifuged at 12,000 g for 20 minutes, and the resulting supernatant was added to a diluent (10 mM phosphate buffered saline (pH 7.8) supplemented with 0.1% BSA) to dilute the supernatant to 1,000-fold. The diluted supernatant was used as a sample “with pretreatment.”

c) Detection with RCA120

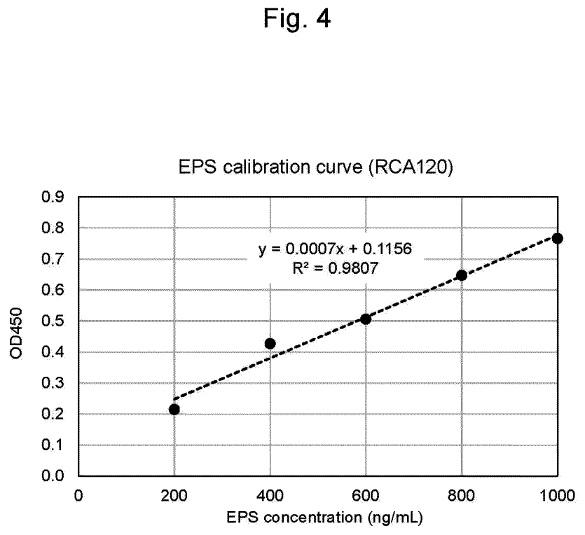

The microtiter plate immobilized with RCA120 and blocked as described in Example 2-2) a) above was washed two times with a wash solution (10 mM phosphate buffered saline (pH 7.8) supplemented with 0.05% Tween 20; the same applies below concerning the detection with RCA120 in this Example), and then each of the diluted EPS standards or the sample with or without pretreatment as described above was added at 100 μl/well, and incubated for reaction at 37° C. for 1 hour. After the plate was washed three times with the wash solution, biotin-labeled RCA120 prepared at 0.1 μg/ml in a diluent (10 mM phosphate buffered saline (pH 7.8)) was added thereto at 100 μl/well, and incubated for reaction at 37° C. for 1 hour. After the microtiter plate was washed three times with the wash solution, peroxidase-labeled streptavidin was added thereto, and incubated for reaction at 37° C. for 15 minutes. After the microtiter plate was washed three times with the wash solution, chromogenic substrate TMB (KPL SureBlue™) was added thereto at 100 μl/well, and incubated at room temperature for 10 minutes to produce a reaction to develop color. Then, 1 M hydrochloric acid was added at 100 μl/well to stop the reaction, and the absorbance at 450 nm was measured using a microplate reader. A calibration curve was prepared based on the measured values (mean values) obtained with the use of the diluted EPS standards (FIG. 4).

On the basis of the calibration curve shown in FIG. 4, the amount of EPS in the sample was determined based on the measured value obtained with the use of the sample with pretreatment or the sample without pretreatment (Table 2).

### 3) Comparison of Results of Quantification

As shown in Table 2, the results of quantification of EPS in the samples (without pretreatment) derived from the culture supernatants were 0.61 mg/ml by the measurement system using ConA and 0.58 mg/ml by the measurement system using RCA120, respectively. The measured values of EPS were approximately equivalent to the results of measurement using samples after removal of proteins from the culture supernatants by TCA treatment (0.55 mg/ml and 0.54 mg/ml, respectively). The results show that, according to the method of the present invention, EPS in a test sample can be measured with superior accuracy, without pretreatment such as protein removal from the test sample.

TABLE 2

| | EPS content (mg/ml) | |
|---|---|---|
| Sample | ConA | RCA120 |
| Sample derived from culture supernatant | 0.61 | 0.58 |
| (without pretreatment) | | |
| Sample derived from culture supernatant | 0.55 | 0.54 |
| (with pretreatment) | | |

### Example 3: Measurement of EPS in Fermented Milk

### 1) Preparation of Fermented Milk

Fermented milk A (hard type) was produced by adding Lactobacillus delbrueckii subsp. bulgaricus OLL1073R-1 strain and Streptococcus thermophilus 1131 strain (which is available as an isolate from Bulgaria Yogurt LB81′, Meiji Co., Ltd.) as starters to a mixture comprising raw milk, skim milk, cream, sugar, and stevia, and putting it into a container, followed by fermentation. Fermented milk B (drink type) was produced by adding Lactobacillus delbrueckii subsp. bulgaricus OLL1073R-1 strain and Streptococcus thermophilus 1131 strain as starters to a mixture comprising raw milk, skim milk, and cream, followed by fermentation in a tank.

### 2) Preparation of Microtiter Plate

ConA prepared at 10 μg/ml in 0.1 M HEPES (pH 8.0) was added at 100 μl/well to a microtiter plate, and the plate was left at 4° C. overnight for immobilization of ConA. Subsequently, the microtiter plate was washed two times with 0.1 M HEPES (pH 8.0), and then a 0.1 M HEPES buffer (pH 8.0) supplemented with 1% BSA was added at 200 μl/well, and the plate was left at 4° C. overnight, for blocking the plate.

### 3) Preparation of Standards and Samples

The EPS standard derived from the OLL1073R-1 strain prepared in Example 1-1) was prepared at 200 to 1,000 ng/ml in a diluent (0.1 M HEPES buffer (pH 8.0) supplemented with 0.1% BSA) to obtain diluted EPS standards.

Fermented milk A and Fermented milk B produced in Example 3-1) were each centrifuged at 12,000 g for 10 minutes, and the supernatants were collected. The collected supernatants were each diluted to 2-fold with 1 M HEPES buffer (pH 8.0) and centrifuged at 12,000 g for 10 minutes, and the supernatants were collected. The supernatants were each diluted to 2-fold with a diluent (1 M HEPES buffer (pH 8.0)), and further diluted with the diluent so as to prepare samples falling within the range of the calibration curve.

### 4) Detection of EPS Using ConA

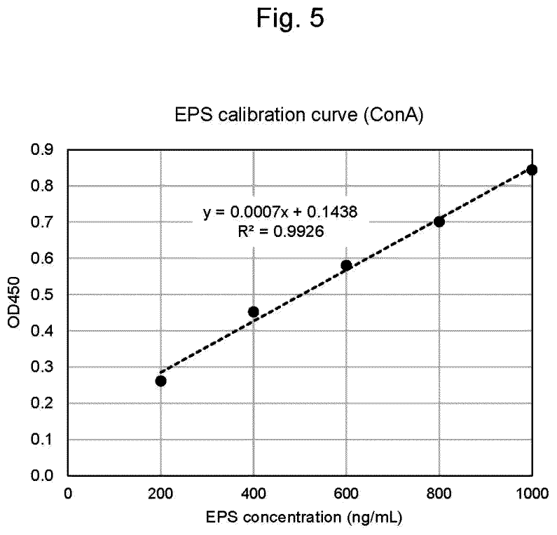

The microtiter plate having ConA immobilized thereon and blocked as described in Example 3-2) above was washed two times with a wash solution (0.1 M HEPES buffer (pH 8.0) supplemented with 0.05% Tween 20; the same applies below concerning the detection with ConA in this Example), and then each of the diluted standards or the sample derived from Fermented milk A or Fermented milk B prepared in Example 3-3) was added at 100 μl/well, and incubated for reaction at 37° C. for 30 minutes. After the plate was washed three times with the wash solution, peroxidase-labeled ConA prepared at 1 μg/ml in a diluent (0.1 M HEPES buffer (pH 8.0)) was added thereto at 100 μl/well, and incubated for reaction at 37° C. for 30 minutes. After the microtiter plate was washed three times with the wash solution, chromogenic substrate TMB (KPL SureBlue™) was added thereto at 100 μl/well, and incubated at room temperature for 10 minutes to produce a reaction to develop color. Then, 1 M hydrochloric acid was added at 100 μl/well to stop the reaction, and the absorbance at 450 nm was measured using a microplate reader. A calibration curve was prepared based on the measured values (mean values) obtained with the use of the diluted EPS standards (FIG. 5).

On the basis of the calibration curve shown in FIG. 5, the amount of EPS in Fermented milk A or Fermented milk B was determined based on the measured value obtained with the use of the sample derived from Fermented milk A or Fermented milk B (Table 3).

TABLE 3

| | EPS content (μg/ml) |
|---|---|
| | ConA |
| Fermented milk A Hard type | 45.9 |
| Fermented milk B Drink type | 67.1 |

The method of the present invention using a lectin was shown to be capable of quantitative detection of EPS in a fermented food.

### Example 4

Fermented milk C (hard type) was produced by adding Lactobacillus delbrueckii subsp. bulgaricus OLL1255 strain (Accession Number: NITE BP-76), Streptococcus thermophilus OLS3294 strain (Accession Number: NITE BP-00077), and Lactobacillus gasseri OLL2716 strain (Accession Number: FERM BP-6999), as starters, to a mixture comprising raw milk, skim milk, cream, sugar, and stevia, and putting it into a container, followed by fermentation.

Lactobacillus delbrueckii subsp. bulgaricus OLL1255 strain is deposited internationally under the Budapest Treaty with the NITE Patent Microorganisms Depositary, the National Institute of Technology and Evaluation (NITE-NPMD) (#122, 2-5-8 Kazusakamatari, Kisarazu-shi. Chiba, 292-0818, Japan) as of Feb. 10, 2005 (the date of the original deposit), under Accession Number NITE BP-76. This deposited strain was transferred from the domestic deposit (the original deposit) to the international deposit under the Budapest Treaty on Apr. 1, 2009. The current depositor of Lactobacillus delbrueckii subsp. bulgaricus OLL1255 strain is Meiji Co., Ltd.

Streptococcus thermophilus OLS3294 strain is deposited internationally with the NITE Patent Microorganisms Depositary, the National Institute of Technology and Evaluation (NITE-NPMD) (#122, 2-5-8 Kazusakamatari, Kisarazu-shi, Chiba, 292-0818, Japan) as of Feb. 10, 2005 (the date of the original deposit). Concerning this deposited strain, the transfer from the domestic deposit (the original deposit: domestic accession number: NITE P-77) to the international deposit under the Budapest Treaty was requested to NITE-NPMD on Mar. 3, 2022 (Provisional Receipt Number: NITE ABP-77), and the receipt of the deposit is to be issued under the Accession Number NITE BP-00077 of the international deposit (as of Feb. 10, 2005 (the date of the original deposit)). The current depositor of Streptococcus thermophilus OLS3294 strain is Meiji Co., Ltd.

Lactobacillus gasseri OLL2716 strain is deposited internationally under the Budapest Treaty with the International Patent Organism Depositary, the National Institute of Technology and Evaluation (NITE-IPOD) (the former names: the National Institute of Bioscience and Human-Technology, Agency of Industrial Science and Technology, Ministry of International Trade and Industry, and as its subsequent name the International Patent Organism Depository, the National Institute of Advanced Industrial Science and Technology) (#120, 2-5-8 Kazusakamatari, Kisarazu-shi, Chiba, 292-0818, Japan) as of May 24, 1999 (the date of the original deposit), under Accession Number FERM BP-6999. This deposited strain was transferred from the domestic deposit (the original deposit) to the international deposit under the Budapest Treaty on Jan. 14, 2000. The current depositor of Lactobacillus gasseri OLL2716 strain is Meiji Co., Ltd.

To 950 μl (1,000 mg) of Fermented milk C, 50 μl of the EPS standard derived from the OLL1073R-1 strain prepared in Example 1-1) (1 mg/ml) or 50 μl of purified water was added and mixed, and then EPS was measured in the same method as in Example 3 with the use of a microtiter plate having ConA immobilized thereon and blocked as prepared in accordance with Example 3.

As a result, the sample supplemented with the EPS standard was found to show a distinctly higher measured value than a sample supplemented with purified water (control), and the EPS concentration corresponding to a difference between the measured value of the sample supplemented with the EPS standard and the measured value of the control sample was very close (99%) to the added amount of the EPS standard (50 μg/ml). While Fermented milk C contained sugar and stevia, the reaction between EPS and ConA was not inhibited by sugar or stevia, and the EPS added to Fermented milk C was successfully measured. Accordingly, the method of the present invention was shown to be capable of quantification of EPS with high reliability.

### Example 5

### 1) Fermented Milk

A commercially available fermented milk drink “Meiji Probio Yogurt R-1, drink type,” in which Meiji Co., Ltd.; Lactobacillus delbrueckii subsp. bulgaricus OLL1073R-1 strain and Streptococcus thermophilus were used, was used as a fermented milk for preparation of assay samples.

### 2) Preparation of Microtiter Plate

ConA prepared at 10 μg/ml in 0.1 M HEPES (pH 8.0) was added at 100 μl/well to a microtiter plate, and the plate was left at 4° C. overnight for immobilization of ConA. Subsequently, the microtiter plate was washed two times with 0.1 M HEPES (pH 8.0), and then a 0.1 M HEPES buffer (pH 8.0) supplemented with 1% BSA was added at 200 μl/well, and the plate was left at 4° C. overnight, for blocking the plate.

### 3) Preparation of Standards and Samples

The EPS standard derived from the OLL1073R-1 strain prepared in Example 1-1) was prepared at 200 to 1,000 ng/ml in a diluent (0.1 M HEPES buffer (pH 8.0) supplemented with 0.1% BSA) to obtain diluted EPS standards.

The fermented milk as described in Example 5-1) was centrifuged at 12,000 g for 10 minutes, and the supernatant was collected. The collected supernatant was diluted to 2-fold with 1 M HEPES buffer (pH 8.0) and centrifuged at 12,000 g for 10 minutes, and the supernatant was collected. Ethanol (99.5%) was added to the collected supernatant to an ethanol concentration of 70%, centrifuged at 12,000 g for 10 minutes, the supernatant was removed, 0.1 M HEPES buffer (pH 8.0) was added to the precipitate to dissolve the precipitate and have a volume equal to that of the assay sample before centrifugation. The resultant was further diluted with the diluent so as to prepare a sample falling within the range of the calibration curve.

### 4) Detection of EPS Using ConA

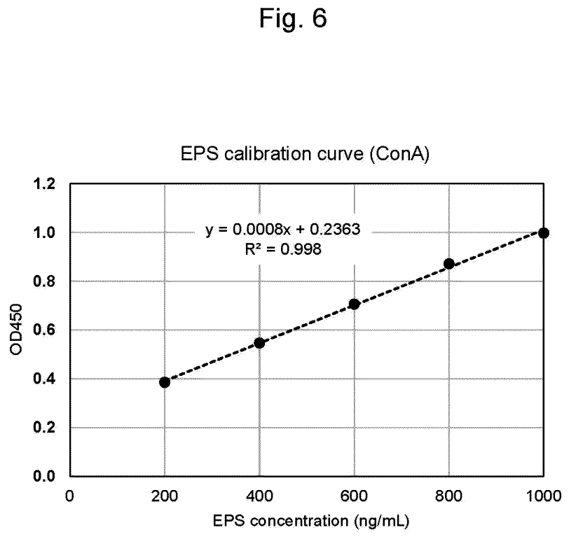

The microtiter plate having ConA immobilized thereon and blocked as described in Example 5-2) above was washed two times with a wash solution (0.1 M HEPES buffer (pH 8.0) supplemented with 0.05% Tween 20), and then each of the diluted standards or the sample derived from the fermented milk as prepared in Example 5-3) was added at 100 μl/well, and incubated for reaction at 37° C. for 30 minutes. After the plate was washed three times with the wash solution, peroxidase-labeled ConA prepared at 0.1 μg/ml in a diluent (0.1 M HEPES buffer (pH 8.0)) was added thereto at 100 μl/well, and incubated for reaction at 37° C. for 30 minutes. After the microtiter plate was washed three times with the wash solution, chromogenic substrate TMB (KPL SureBlue™) was added thereto at 100 μl/well, and incubated at room temperature for 10 minutes to produce a reaction to develop color. Then, 1 M hydrochloric acid was added at 100 μl/well to stop the reaction, and the absorbance at 450 nm was measured using a microplate reader. A calibration curve was prepared based on the measured values (mean values) obtained with the use of the diluted EPS standards (FIG. 6).

On the basis of the calibration curve shown in FIG. 6, the amount of EPS in the fermented milk was determined based on the measured value obtained with the use of the above-mentioned sample derived from the fermented milk (Table 4).

TABLE 4

| | EPS content (μg/ml) |
|---|---|
| | ConA |
| Meiji Probio Yogurt R-1, drink type | 32.2 |

The method of the present invention using a lectin was shown to be capable of quantitative detection of EPS in a fermented food.

### Example 6

The test of EPS addition and collection was performed in the same method as in Example 4, except that the standards and the samples were prepared in accordance with Example 5-3).

To 950 μl (1,000 mg) of a commercially available fermented milk drink “Meiji Probio Yogurt LG21, drink type” (Meiji Co., Ltd.; Lactobacillus delbrueckii subsp. bulgaricus, Lactobacillus gasseri OLL2716 strain and Streptococcus thermophilus were used therein), 50 μl of the EPS standard derived from the OLL1073R-1 strain prepared in Example 1-1) (1 mg/ml) or 50 μl of purified water was added and mixed, and then EPS was measured in the same method as in Example 5 with the use of a microtiter plate having ConA immobilized thereon and blocked as prepared in accordance with Example 5-2.

As a result, the sample supplemented with the EPS standard was found to show a distinctly higher measured value than a sample supplemented with purified water (control), and the EPS concentration corresponding to a difference between the measured value of the sample supplemented with the EPS standard and the measured value of the control sample was very close (100.4%) to the added amount of the EPS standard (50 μg/ml). The fermented milk drink used in the present Example contains glucose-fructose syrup. Glucose is known to inhibit a reaction of ConA. According to the method described above, however, the reaction between EPS and ConA was not inhibited, and EPS added to the fermented milk drink was successfully measured. Accordingly, the method of the present invention was found to be capable of quantification of EPS with high reliability.

### INDUSTRIAL APPLICABILITY

The present invention can be used advantageously for rapid, simple and high sensitive detection or quantification of an exopolysaccharide (EPS). The present invention can provide, for example, an assay method comprising sandwiching EPS in a culture solution or fermented milk between lectins and then quantifying EPS by enzyme reaction in a rapid and simple method with high sensitivity. The present invention can be used advantageously for providing, in particular, a method for simple and high sensitive detection or quantification of EPS even in a culture such as a fermented product, which is rich in impurity sugars such as monosaccharides or disaccharides and is thus likely to cause a high background signal.

All publications, patents, and patent applications cited herein are incorporated herein by reference in their entirety.

### DESCRIPTION OF NUMERAL REFERENCES

1: First lectin; 2: Second lectin (labeled lectin); 3: Exopolysaccharide; 4: Lectin-binding sugar; 5: Impurity sugar; 6: Label; 7: Sample

1. A method for detecting an exopolysaccharide comprising:
contacting a sample containing an exopolysaccharide (EPS) with (i) a lectin capable of binding specifically to the exopolysaccharide and (ii) a labeled lectin capable of binding specifically to the exopolysaccharide, and
detecting an exopolysaccharide bound to both the lectin of (i) and the labeled lectin of (ii) using a label of the labeled lectin.

2. The method according to claim 1, wherein the sample contains a culture or a fraction thereof comprising a microorganism having an ability to produce the exopolysaccharide.

3. The method according to claim 2, wherein the microorganism is a lactic acid bacterium or a bifidobacterium.

4. The method according to claim 1, wherein the exopolysaccharide is from a lactic acid bacterium or a bifidobacterium.

5. The method according to claim 3, wherein the lactic acid bacterium is Lactobacillus delbrueckii subsp. bulgaricus OLL1073R-1 strain (Accession Number: FERM BP-10741).

6. The method according claim 1, wherein the labeled lectin of (ii) comprises a lectin that is the same as the lectin of (i).

7. The method according to claim 1, wherein the lectin of (i) and the labeled lectin of (ii) bind specifically to a sugar in the exopolysaccharide.

8. The method according to claim 7, wherein the sugar is or comprises galactose or glucose.

9. The method according to claim 1, wherein the lectin of (i) and the labeled lectin of (ii) are RCA120 or concanavalin A.

10. The method according to claim 1, wherein the lectin of (i) is immobilized on a solid carrier.

11. The method according to claim 10, wherein the solid carrier is washed before the detection.

12. The method according to claim 2, wherein the culture is a fermented food.

13. The method according to claim 12, wherein the fermented food is fermented milk.

14. The method according to claim 1, which is a method for quantitatively detecting an exopolysaccharide comprising determining the content of an exopolysaccharide in a sample based on the results of the detection of the exopolysaccharide.

15. A method for producing a fermented food comprising:
performing fermentation under conditions suitable for increasing an exopolysaccharide, while quantifying an exopolysaccharide by the method according to claim 14, and
increasing the content of the exopolysaccharide in a fermented food.
